(19) United States
(12) Patent Application Publication
(10) Pub. No.: US 2009/0098206 A1
(43) Pub. Date: Apr. 16, 2009

(54) Particulate Metal Oxide

(75) Inventors: Lorna Margaret Kessell, North Yorkshire (GB); Ian Robert Tooley, Cleveland (GB)

Correspondence Address: JONES DAY, 222 EAST 41ST ST, NEW YORK, NY, 10017, US

(73) Assignee: Croda International PLC, Goole, North Humberside (GB)

(21) Appl. No.: 12/086,859

(22) PCT Filed: Dec. 21, 2006
(86) PCT No.: PCT/GB2006/004842

§ 371 Date: Oct. 7, 2008

(30) Foreign Application Priority Data
Dec. 23, 2005 (GB) 0526328.0

(51) Int. Cl. A61K 9/14; C07F 7/28; A61K 8/04; A61K 33/24; A61Q 17/04

(52) U.S. Cl. 424/489; 556/51; 424/59; 424/617

(57) ABSTRACT

A particulate metal oxide having a median volume particle diameter in the range from 24 to 42 nm, a photogreying index in the range from 0.05 to 3 and/or and a yellowing index of less than 6. The dispersion can be used in a sunscreen product which is transparent, exhibits effective UV protection, reduced photoactivity, and reduced yellowing in combination with organic UV absorbers.

### FIELD OF INVENTION

The present invention relates to metal oxide particles, a metal oxide dispersion, and in particular to the use thereof in a sunscreen product.

### BACKGROUND

Metal oxides such as titanium dioxide, zinc oxide and iron oxide have been employed as attenuators of ultraviolet light in sunscreens. Due to the increased awareness of the link between ultraviolet light and skin cancer, there has been a requirement for ultraviolet light protection in everyday skincare and cosmetics products. There is a requirement for a metal oxide in a form which when Incorporated into sunscreen products exhibits both effective UV absorption properties and be transparent in use. Metal oxides are often used in sunscreen products in combination with organic attenuators of ultraviolet light. Unfortunately, metal oxides may form complexes with UV absorbers which can result in undesirable yellowing of end use sunscreen products.

In addition, metal oxides may be photoactive which can result in unwanted greying of end use sunscreen products.

Thus, there is a need to provide a metal oxide which is transparent, exhibits effective UV absorption properties, reduced photoactivity, and reduced yellowing in combination with organic sunscreens.

### SUMMARY OF THE INVENTION

We have now surprisingly discovered an improved metal oxide, which overcomes or significantly reduces at least one of the aforementioned problems.

Accordingly, the present invention provides a particulate metal oxide having a median volume particle diameter in the range from 24 to 42 nm and a photogreying index in the range from 0.05 to 3.

The present invention also provides a particulate metal oxide having a median volume particle diameter in the range from 24 to 42 nm and a yellowing index of less than 6.

The present invention further provides a dispersion comprising metal oxide particles having an extinction coefficient at 524 nm in the range from 0.4 to 1.5 l/g/cm, a photogreying index in the range from 0.05 to 3, and a yellowing index of less than 6.

The present invention still further provides the use of metal oxide particles having a median volume particle diameter in the range from 24 to 42 nm and a photogreying index in the range from 0.05 to 3 to produce a sunscreen having reduced photoactivity.

The present invention yet further provides the use of a dispersion comprising particles of metal oxide having a median volume particle diameter in the range from 24 to 42 nm and a yellowing index of less than 6, in the manufacture of a transparent sunscreen comprising an organic UV absorber.

Preferably the metal oxide used in the present invention comprises an oxide of titanium, zinc or iron, and most preferably the metal oxide is titanium dioxide.

The preferred titanium dioxide particles comprise anatase and/or rutile crystal form. The titanium dioxide in the particles suitably comprises a major portion of rutile, preferably greater than 70%, more preferably greater than 80%, particularly greater than 90%, and especially greater than 95% by weight of rutile.

The basic particles may be prepared by standard procedures, such as using the chloride process, or by the sulphate process, or by hydrolysis of an appropriate titanium compound such as titanium oxydichloride or an organic or Inorganic titanate, or by oxidation of an oxidisable titanium compound, e.g. in the vapour state. The titanium dioxide particles are preferably prepared by the hydrolysis of a titanium compound, particularly of titanium oxydichloride.

The particles of metal oxide according to the present invention are preferably coated with silica. The amount of silica coating is suitably in the range from 5% to 25%, preferably 7% to 20%, more preferably 8% to 15%, particularly 9% to 12%, and especially 10% to 11% by weight, calculated with respect to the weight of metal oxide core particles. The silica coating may be applied using techniques known in the art. A typical process comprises forming an aqueous dispersion of metal oxide particles in the presence of a soluble salt of silica. This dispersion is preferably alkali, more preferably having a pH of greater than 8, particularly in the range from 9 to 12. The precipitation of the silica is achieved by adjusting the pH of the dispersion by the addition of acid or alkali, as appropriate.

The particles of metal oxide used in the present invention are preferably hydrophobic. The hydrophobicity of the metal oxide can be determined by pressing a disc of metal oxide powder, and measuring the contact angle of a drop of water placed thereon, by standard techniques known in the art. The contact angle of a hydrophobic metal oxide is preferably greater than 50°.

The metal oxide particles are preferably coated in order to render them hydrophobic. Suitable coating materials are water-repellent, preferably organic, and include fatty acids, preferably fatty acids containing 10 to 20 carbon atoms, such as lauric add, stearic acid and isostearic acid, salts of the above fatty adds such as sodium salts and aluminium salts, fatty alcohols, such as stearyl alcohol, and silicones such as polydimethylsiloxane and substituted polydimethylsiloxanes, and reactive silicones such as methylhydrosiloxane and polymers and copolymers thereof. Stearic add and/or salt thereof. Is particularly preferred. The organic coating may be applied using any conventional process. Typically, metal oxide particles are dispersed in water and heated to a temperature in the range from 50° C. to 80° C. A fatty add, for example, is then deposited on the metal oxide particles by adding a salt of the fatty acid (e.g. sodium stearate) to the dispersion, followed by an acid. Alternatively, the metal oxide particles can be mixed with a solution of the water-repellent material in an organic solvent, followed by evaporation of the solvent. In an alternative embodiment of the invention, the water-repellant material can be added directly to the composition according to the present invention, during preparation thereof, such that the hydrophobic coating is formed in situ. Generally, the particles are treated with up to 25%, suitably in the range from 5% to 20%, more preferably 11% to 16%, particularly 12% to 15%, and especially 13% to 14% by weight of organic material, preferably fatty acid, calculated with respect to the metal oxide core particles.

In a preferred embodiment of the invention, the metal oxide particles are coated with both an inorganic silica and an organic coating, either sequentially or as a mixture. It is preferred that the silica is applied first followed by the organic coating, preferably fatty acid and/or salt thereof. Thus, preferred metal oxide particles for use in the present invention comprise (i) in the range from 70% to 94%, more preferably 75% to 87%, particularly 78% to 84%, and especially 80% to 82% by weight of metal oxide, preferably titanium dioxide, with respect to the total weight of the particles, (ii) in the range from 2% to 12%, more preferably 5% to 11%, particularly 7% to 10%, and especially 8% to 9% by weight of silica coating, with respect to the total weight of the particles, and (iii) in the range from 4% to 18%, more preferably 7% to 15%, particularly 9% to 12%, and especially 10% to 11% by weight of organic coating, preferably fatty acid and/or salt thereof, with respect to the total weight of the particles.

The individual or primary metal oxide particles are preferably acicular in shape and have a long axis (maximum dimension or length) and short axis (minimum dimension or width). The third axis of the particles (or depth) is preferably approximately the same dimensions as the width.

The mean length by number of the primary metal oxide particles is suitably in the range from 50 to 90 nm, preferably 55 to 77 nm, more preferably 55 to 73 nm, particularly 60 to 70 nm, and especially 60 to 65 nm. The mean width by number of the particles is suitably in the range from 5 to 20 nm, preferably 8 to 19 nm, more preferably 10 to 18 nm, particularly 12 to 17 nm, and especially 14 to 16 nm. The primary titanium dioxide particles preferably have a mean aspect ratio d₁:d₂ (where d₁ and d₂, respectively, are the length and width of the particle) in the range from 2.0 to 8.0:1, more preferably 3.0 to 6.5:1, particularly 4.0 to 6.0:1, and especially 4.5 to 5.5:1. The size of the primary particles can be suitably measured using electron microscopy. The size of a particle can be determined by measuring the length and width of a filler particle selected from a photographic image obtained by using a transmission electron microscope.

The metal oxide particles suitably have a mean crystal size (measured by X-ray diffraction as herein described) In the range from 4 to 10 nm, preferably 5 to 9 nm, more preferably 5.5 to 8.5 nm, particularly 6 to 8 nm, and especially 6.6 to 7.5 nm.

The size distribution of the crystal size of the metal oxide particles can be important, and suitably at least 30%, preferably at least 40%, more preferably at least 50%, particularly at least 60%, and especially at least 70% by weight of the metal oxide particles have a crystal size within one or more of the above preferred ranges for the mean crystal size.

When formed into a dispersion according to the present invention, the particulate metal oxide suitably has a median volume particle diameter (equivalent spherical diameter corresponding to 50% of the volume of all the particles, read on the cumulative distribution curve relating volume % to the diameter of the particles—often referred to as the “D(v,0.5)” value)) (hereinafter referred to as dispersion particle size), measured as herein described, in the range from 24 to 42 nm, preferably 27 to 39 nm, more preferably 29 to 37 nm, particularly 31 to 35 nm, and especially 32 to 34 nm.

The size distribution of the metal oxide particles in dispersion can also be an important parameter in obtaining, for example, a sunscreen product having the required properties. In a preferred embodiment suitably less than 10% by volume of metal oxide particles have a volume diameter of more than 13 nm, preferably more than 11 nm, more preferably more than 10 nm, particularly more than 9 nm, and especially more than 8 nm below the median volume particle diameter. In addition, suitably less than 16% by volume of metal oxide particles have a volume diameter of more than 11 nm, preferably more than 9 nm, more preferably more than 8 nm, particularly more than 7 nm, and especially more than 6 nm below the median volume particle diameter. Further, suitably less than 30% by volume of metal oxide particles have a volume diameter of more than 7 nm, preferably more than 6 nm, more preferably more than 5 nm, particularly more than 4 nm, and especially more than 3 nm below the median volume particle diameter.

Also, suitably more than 90% by volume of metal oxide particles have a volume diameter of less than 30 nm, preferably less than 27 nm, more preferably less than 25 nm, particularly less than 23 nm, and especially less than 21 nm above the median volume particle diameter. In addition, suitably more than 84% by volume of metal oxide particles have a volume diameter of less than 19 nm, preferably less than 18 nm, more preferably less than 17 nm, particularly less than 16 nm, and especially less than 15 nm above the median volume particle diameter. Further, suitably more than 70% by volume of metal oxide particles have a volume diameter of less than 8 nm, preferably less than 7 nm, more preferably less than 6 nm, particularly less than 5 nm, and especially less than 4 nm above the median volume particle diameter.

Dispersion particle size of the metal oxide particles described herein may be measured by electron microscopy, coulter counter, sedimentation analysis and static or dynamic light scattering. Techniques based on sedimentation analysis are preferred. The median particle size may be determined by plotting a cumulative distribution curve representing the percentage of particle volume below chosen particle sizes and measuring the 50th percentile. The median particle volume diameter and particle size distribution of the metal oxide particles in dispersion is suitably measured using a Brookhaven particle sizer, as described herein.

In a particularly preferred embodiment of the Invention, the metal oxide particles have a BET specific surface area, measured as described herein, of greater than 40, more preferably in the range from 50 to 100, particularly 60 to 90, and especially 65 to 75 m²/g.

The metal oxide particles used in the present invention are transparent, suitably having an extinction coefficient at 524 nm (E₂₅₄), measured as herein described, In the range from 0.4 to 1.5, preferably 0.6 to 1.4, more preferably 0.7 to 1.3, particularly 0.8 to 1.2, and especially 0.9 to 1.1 l/g/cm. In addition, the metal oxide particles suitably have an extinction coefficient at 450 nm (E₄₅₀), measured as herein described, in the range from 0.8 to 2.2, preferably 1.0 to 2.0, more preferably 1.2 to 1.8, particularly 1.3 to 1.7, and especially 1.4 to 1.6 l/g/cm.

The metal oxide particles exhibit effective UV absorption, suitably having an extinction coefficient at 360 nm (E₃₆₀), measured as herein described, in the range from 2 to 14, preferably 3 to 10, more preferably 4 to 8, particularly 5 to 7, and especially 5.5 to 6.5 l/g/cm. The metal oxide particles also suitably have an extinction coefficient at 308 nm (E₃₀₈), measured as herein described, in the range from 38 to 52, preferably 40 to 50, more preferably 42 to 48, particularly 43 to 47, and especially 44 to 46 l/g/cm.

The metal oxide particles suitably have a maximum extinction coefficient E(max), measured as herein described, in the range from 55 to 75, preferably 59 to 71, more preferably 61 to 69, particularly 63 to 67, and especially 64 to 66 l/g/cm. The metal oxide particles suitably have a λ(max), measured as herein described, in the range from 265 to 285, preferably 269 to 281, more preferably 271 to 279, particularly 273 to 277, and especially 274 to 276 nm.

The metal oxide particles suitably exhibit reduced whiteness, having a change in whiteness ΔL of a sunscreen product containing the particles, measured as herein described, of less than 4, preferably In the range from 0.5 to 3, more preferably 1.2 to 2.7, and particularly 1.7 to 2.4. In addition, a sunscreen product containing the particles preferably has a whiteness index, measured as herein described, of less than 100%, more preferably in the range from 10% to 80%, particularly 20% to 60%, and especially 30% to 50%.

A particularly surprising feature of the present invention is that the metal oxide particles have significantly reduced photoactivity, suitably having a photogreying index, measured as herein described, of less than 5, preferably in the range from 0.05 to 3, more preferably 0.2 to 2, particularly 0.5 to 1.5, and especially 0.7 to 0.95. Photogreying is an indirect measure of the quality of the coating layer on the metal oxide core particles, and lower values indicate improved coating coverage such as more complete surface coverage, increased thickness and/or greater density of the coating layer.

A further surprising feature of the present invention is the improved compatability, i.e. reduced yellowing, of the metal oxide particles when present in combination with organic UV absorbers. The metal oxide particles suitably have a yellowing index, measured as herein described, of less than 6, preferably in the range from 0.5 to 5, more preferably 1 to 4, particularly 1.5 to 3, and especially 2 to 2.5.

The particulate metal oxide according to the present invention may be in the form of a free-flowing powder. A powder having the required particle size for the secondary metal oxide particles, as described herein, may be produced by milling processes known in the art. The final milling stage of the metal oxide is suitably carried out in dry, gas-borne conditions to reduce aggregation. A fluid energy mill can be used in which the aggregated metal oxide powder is continuously injected into highly turbulent conditions in a confined chamber where multiple, high energy collisions occur with the walls of the chamber and/or between the aggregates. The milled powder is then carried into a cyclone and/or bag filter for recovery. The fluid used in the energy mill may be any gas, cold or heated, or superheated dry steam.

The particulate metal oxide may be formed into a slurry, or preferably a liquid dispersion, in any suitable aqueous or organic liquid medium. By liquid dispersion is meant a true dispersion, i.e. where the solid particles are stable to aggregation. The particles in the dispersion are relatively uniformly dispersed and resistant to settling out on standing, but if some settling out does occur, the particles can be easily redispersed by simple agitation.

Cosmetically acceptable materials are preferred as the liquid medium. A useful organic medium is a liquid oil such as vegetable oils, e.g. fatty acid glycerides, fatty add esters and fatty alcohols. A preferred organic medium is a siloxane fluid, especially a cyclic oligomeric dialkylsiloxane, such as the cyclic pentamer of dimethylsiloxane known as cyclomethicone. Alternative fluids include dimethylsiloxane linear oligomers or polymers having a suitable fluidity and phenyltris(trimethylsiloxy)silane (also known as phenyltrimethicone).

Examples of suitable organic media include non-polar materials such as C13-14 isoparaffin, isohexadecane, paraffinum liquidum (mineral oil), squalane, squalene, hydrogenated polyisobutene, and polydecene; and polar materials such as C12-15 alkyl benzoate, caprylic/capric triglyceride, cetearyl isononanoate, ethylhexyl isostearate, ethylhexyl palmitate, isononyl isononanoate, isopropyl isostearate, isopropyl myristate, isostearyl isostearate, isostearyl neopentanoate, octyldodecanol, pentaerythrityl tetraisostearate, PPG-15 stearyl ether, triethylhexyl triglyceride, dicaprylyl carbonate, ethylhexyl stearate, helianthus annus (sunflower) seed oil, isopropyl palmitate, and octyldodecyl neopentanoate.

The dispersion according to the present invention may also contain a dispersing agent in order to improve the properties thereof. The dispersing agent is suitably present in the range from 1% to 30%, preferably 2% to 20%, more preferably 9% to 20%, particularly 11% to 17%, and especially 13% to 15% by weight based on the total weight of metal oxide particles.

Suitable dispersing agents include substituted carboxylic acids, soap bases and polyhydroxy acids. Typically the dispersing agent can be one having a formula X.CO.AR in which A is a divalent bridging group, R is a primary secondary or tertiary amino group or a salt thereof with an acid or a quaternary ammonium salt group and X is the residue of a polyester chain which together with the O group is derived from a hydroxy carboxylic acid of the formula HO—R′—COOH. As examples of typical dispersing agents are those based on ricinoleic acid, hydroxystearic acid, hydrogenated castor oil fatty acid which contains in addition to 12-hydroxystearic acid small amounts of stearic acid and palmitic add. Dispersing agents based on one or more polyesters or salts of a hydroxycarboxylic acid and a carboxylic acid free of hydroxy groups can also be used. Compounds of various molecular weights can be used.

Other suitable dispersing agents are those monoesters of fatty acid alkanolamides and carboxylic acids and their salts. Alkanolamides are based on ethanolamine, propanolamine or aminoethyl ethanolamine for example. Alternative dispersing agents are those based on polymers or copolymers of acrylic or methacrylic acids, e.g. block copolymers of such monomers. Other dispersing agents of similar general form are those having epoxy groups in the constituent radicals such as those based on the ethoxylated phosphate esters. The dispersing agent can be one of those commercially referred to as a hyper dispersant. Polyhydroxystearic add is a particularly preferred dispersing agent.

An advantage of the present invention is that dispersions can be produced which contain at least 35%, preferably at least 40%, more preferably at least 45%, particularly at least 50%, especially at least 55%, and generally up to 60% by weight of the total weight of the dispersion, of metal oxide particles.

A composition, preferably a sunscreen product, containing the metal oxide particles according to the present invention suitably has a Sun Protection Factor (SPF), measured as herein described, of greater than 10, preferably greater than 15, more preferably greater than 20, particularly greater then 25, and especially greater than 30 and up to 40.

The metal oxide particles and dispersions of the present invention are useful as ingredients for preparing sunscreen compositions, especially in the form of emulsions. The compositions may further contain conventional additives suitable for use in the intended application, such as conventional cosmetic ingredients used in sunscreens. The particulate metal oxide as defined herein, may provide the only ultraviolet light attenuators in a sunscreen product according to the invention, but other sunscreening agents, such as other metal oxides and/or other organic materials may also be added. For example, the preferred titanium dioxide particles defined herein may be used in combination with other existing commercially available titanium dioxide and/or zinc oxide sunscreens.

The metal oxide particles and dispersions described herein are particularly suitable for using in combination with organic UV absorbers such as butyl methoxydibenzoylmethane (avobenzone), benzophenone-3 (oxybenzone), 4-methylbenzylidene camphor (enzacamene), benzophenone-4 (sulisobenzone), bis-ethylhexyloxyphenol methoxyphenyl triazine (bemotrizinol), diethylamino hydroxybenzoyl hexyl benzoate, diethylhexyl butamido triazone, disodium phenyl dibenzimidazole tetrasulfonate, drometrizole trisiloxane, ethylhexyl dimethyl PABA (padimate O), ethylhexyl methoxycinnamate (octinoxate), ethylhexyl salicylate (octisalate), ethylhexyl triazone, homosalate, Isoamyl p-methoxycinnamate (amiloxate), isopropyl methoxycinnamate, menthyl anthranilate (meradimate), methylene bis-benzotriazolyl tetramethylbutylphenol (bisoctrizole), octocrylene, PABA (aminobenzoic acid), phenylbenzimidazole sulfonic add (ensulizole), terephthalylidene dicamphor sulfonic acid, and mixtures thereof. Preferred organic UV absorbers are butyl methoxydibenzoylmethane and benzophenone-3, and particularly butyl methoxydibenzoylmethane.

In this specification the following test methods have been used:

### 1) Crystal Size Measurement of Metal Oxide Particles

Crystal size was measured by X-ray diffraction (XRD) line broadening. Diffraction patterns were measured with Cu Kα radiation in a Siemens D5000 diffractometer equipped with a Sol-X energy dispersive detector acting as a monochromator. Programmable slits were used to measure diffraction from a 12 mm length of specimen with a step size of 0.02° and step counting time of 3 sec. The data was analysed by fitting the diffraction pattern between 22 and 48° 2θ with a set of peaks corresponding to the reflection positions for ruble and, where anatase was present, an additional set of peaks corresponding to those reflections. The fitting process allowed for removal of the effects of instrument broadening on the diffraction line shapes. The value of the weight average mean crystal size was determined for the rutile 110 reflection (at approximately 27.4° 2θ) based on its integral breadth according to the principles of the method of Stokes and Wilson (B. E. Warren, “X-Ray Diffraction”, Addison-Wesley, Reading, Mass., 1969, pp 254-257).

### 2) Median Particle Volume Diameter and Particle Size Distribution of Metal Oxide Particles in Dispersion

A dispersion of metal oxide particles was produced by mixing 6.3 g of polyhydroxystearic acid with 48.7 g of C12-C15 alkylbenzoate, and then adding 45 g of metal oxide into the solution. The mixture was passed through a horizontal bead mill, operating at approximately 2100 r.p.m. and containing zirconia beads as grinding media for 15 minutes. The dispersion of metal oxide particles was diluted to between 30 and 40 g/l by mixing with isopropyl myristate containing 1% by weight of polyhydroxystearic add (it is necessary to ensure that the diluted dispersion is stable prior to measuring particle size (if required, the amount of polyhydroxystearic acid can be adjusted accordingly)). The diluted sample was analysed on the Brookhaven BI-XDC particle sizer in centrifugation mode, and the median particle volume diameter and particle size distribution measured.

### 3) BET Specific Surface Area of Metal Oxide Particles

The single point BET specific surface area was measured using a Micromeritics Flowsorb II 2300.

### 4) Change in Whiteness and Whiteness Index

A sunscreen formulation was coated on to the surface of a glossy black card and drawn down using a No 2 K bar to form a film of 12 microns wet thickness. The film was allowed to dry at room temperature for 10 minutes and the whiteness of the coating on the black surface (L_{F}) measured using a Minolta CR300 colourimeter. The change in whiteness ΔL was calculated by subtracting the whiteness of the substrate (L_{S}) from the whiteness of the coating (L_{F}). The whiteness index is the percentage change in whiteness ΔL compared to a standard titanium dioxide (=100% value) (Tayca MT100T (ex Tayca Corporation)).

### 5) Photogreying Index

A metal oxide dispersion was prepared by milling 15 g of metal oxide powder into 85 g of C12-15 alkyl benzoate for 15 min at 5000 rpm with a mini-motor mill (Elger Torrance MK M50 VSE TFV), 70% filled with 0.8-1.25 mm zirconia beads (ER120SWIDE). Freshly milled dispersions were loaded into a 16 mm diameter×3 mm deep recess in 65×30×6 mm acrylic cells. A quartz glass cover slip was placed over the sample to eliminate contact with the atmosphere, and secured In place by a brass catch. Up to 12 cells could be placed on a rotating platform, positioned 12 cm from a 75 W UV light source (Philips HB 171/A with 4 TL29D16/09N lamps) and Irradiated for 120 minutes. Sample colour (L*a*b* value) was recorded by a commercial colour meter (Minoita chroma meter CR-300), previously calibrated with a standard white tile (L*=97.95). The change in whiteness ΔL* was calculated by subtracting the whiteness of the substrate before exposure to UV light (L*ᵢₙᵢₜᵢₐₗ) from the whiteness of the substrate after exposure to UV light. The photogreying index ΔL*=L*₍ᵢₙᵢₜᵢₐₗ₎−L*₍₁₂₀ₘᵢₙ₎.

### 6) Yellowing Index

23.75 g of the metal oxide dispersion produced in 5) above was thoroughly mixed with 1.75 g of butyl methoxydibenzoylmethane (avobenzone (Parsol 1789)). Sample colour (L*a*b* value) was recorded after approximately one hour by a commercial colour meter (Minolta chroma meter CR-300), previously calibrated with a standard white tile (L*=97.95). The yellowing index Δb*=b*_{(metal codde+Avobenzone)}−b*_{(metal codde)}.

### 7) Sun Protection Factor

The Sun Protection Factor (SPF) of a sunscreen formulation was determined using the In vitro method of Diffey and Robson, J. Soc. Cosmet. Chem. Vol. 40, pp 127-133, 1989.

### 8) Extinction Coefficients

0.1 g sample of a metal oxide dispersion was diluted with 100 ml of cyclohexane. This diluted sample was then further diluted with cyclohexane in the ratio sample:cyclohexane of 1:19. The total dilution was 1:20,000. The diluted sample was then placed in a spectrophotometer (Perkin-Elmer Lambda 2 UV/VIS Spectrophotometer) with a 1 cm path length and the absorbance, of UV and visible light measured. Extinction coefficients were calculated from the equation A=E.c.l, where A=absorbance, E=extinction coefficient in litre per gram per cm, c=concentration in grams per litre, and l=path length in cm.

The invention is Illustrated by the following non-limiting examples.

### EXAMPLES

### Example 1

1 mole of titanium oxydichloride in acidic solution was reacted with 3 moles of NaOH in aqueous solution. After the initial reaction period, the temperature was increased to above 70° C., and stirring continued. The reaction mixture was neutralised by the addition of aqueous NaOH, and allowed to cool below 70° C. The resultant slurry was heated to 50±2° C. and pH adjusted to >9 by addition of 20% NaOH. Sodium silicate was added dropwise, equivalent to 10.5% by weight of SiO₂ on TiO₂ into the agitated slurry. The temperature was raised to 75° C., and the alkali slurry was stirred for 15 min. 13.5% by weight of sodium stearate on TiO₂ dissolved in water was added into the solution. The slurry was equilibrated for 15 minutes and neutralized by adding 20% hydrochloric acid dropwise over 30 minutes before the slurry was allowed to cool to less than 50° C. The slurry was filtered using a Buchner filter until the cake conductivity at 100 gdm⁻³ in water was <150 μS. The filter cake was oven-dried for 16 hours at 110° C. and ground into a fine powder by an IKA Werke dry powder mill operating at 3250 rpm.

A dispersion was produced by mixing 6.3 g of polyhydroxystearic acid with 48.7 g of C12-C15 alkylbenzoate, and then adding 45 g of pre-dried coated titanium dioxide powder produced above into the mixture. The mixture was passed through a horizontal bead mill, operating at 1500 r.p.m. and containing zirconia beads as grinding media for 15 minutes.

The dispersion was subjected to the test procedures described herein, and the titanium dioxide exhibited the following properties:

### i) Extinction Coefficients;

| E₅₂₄ | E₄₅₀ | E₃₀₈ | E₃₆₀ | E(max) | λ (max) |
|---|---|---|---|---|---|
| 1.0 | 1.5 | 45.0 | 6.0 | 65.0 | 275 |

ii) Photogreying index=0.8.
iii) Yellowing index=2.7.

### Example 2

The titanium dioxide dispersion produced in Example 1 was used to prepare a water-in-oil sunscreen emulsion having the following composition:

| | % w/w |
|---|---|
| Phase A | |
| ARLACEL ™ P135 (ex Uniqema) | 2.0 |
| ARLAMOL ™ HD (ex Uniqema) | 5.0 |
| DC 245 | 5.6 |
| ARLAMOL ™ E (ex Uniqema) | 2.4 |
| Jojoba Oil | 3.5 |
| Candelilla Wax | 1.0 |
| Magnesium Stearate | 0.7 |
| Avobenzone | 2.0 |
| Disodium EDTA | 0.1 |
| Titanium Dioxide Dispersion | 11.1 |
| Phase B. | |
| Germaben II | 1.0 |
| Water; Pure | 59.5 |
| Glycerine BP | 4.0 |
| MgSO₄•7H₂O | 0.7 |

The ingredients of phase A were mixed together and heated to 70-80° C. Phase B was mixed together, heated to 70-80° C. and mixed with phase A at 400 rpm. The resulting mixture was homogenised by an Ultra Turrax operating at 12,000 rpm for 2 minutes. Finally, the mixture was allowed to cool to room temperature with intensive stirring.

The formulation was allowed to stabilise and the sample colour (L*a*b* value) was recorded by a commercial colour meter (Minolta chroma meter CR-300), previously calibrated with a standard white file (L*=97.95). The yellowing of the formulation was taken as b* according to the L*a*b* colour range, and the b* value was 3.1

### Example 3

The procedure of Example 2 was repeated except that the formulation contained 2% benzophenone-3 instead of 2% avobenzone. The b* value was 3.5.

### Example 4

The titanium dioxide dispersion produced in Example 1 was used to prepare an oil-in-water sunscreen emulsion having the following composition:

| | % w/w |
|---|---|
| Phase A | |
| ARLACEL ™ 165 (ex Uniqema) | 6.0 |
| SPAN ™ 60 (ex Uniqema) | 0.5 |
| TWEEN ™ 60V (ex Uniqema) | 2.7 |
| Stearyl Alcohol | 1.0 |
| Light Mineral Oil | 8.0 |
| Sweet Almond Oil | 2.0 |
| DC 200 Fluid (350 cs) | 2.0 |
| ESTOL ™ 1543 (ex Uniqema) | 2.0 |
| Avobenzone | 2.0 |
| Disodium EDTA | 0.1 |
| Antaron V-220 | 1.0 |
| Phase B | |
| Titanium Dioxide Dispersion | 11.1 |
| Phase C | |
| Water; Pure | 56.5 |
| Keltrol RD | 0.1 |
| Propylene Glycol | 4.0 |
| Phase D | |
| Phenonip | 1.0 |

Phase C was prepared by dispersing Keltrol in water, and when fully dispersed the propylene glycol was added. Phase C was heated to 70° C. The ingredients of phase A were mixed together and heated to 70° C. Phase B was added to phase A with hand stirring. The resulting mixture was homogenised by an Ultra Turrax operating at 8,000 rpm for 2 minutes. The mixture was then added to phase C with homogenisation (Ultra Turrax, 8,000 rpm). Mixing was continued for a further 2 minutes (Ultra Turrax, 12,000 rpm). The mixture was cooled with moderate stirring. Phase D was added at a temperature of approximately 45° C.

The yellowing of the formulation was measured as described in Example 2, and the b* value was 1.6.

The above examples illustrate the improved properties of a particulate metal oxide, dispersion and sunscreen product according to the present invention.

1. A particulate metal oxide having a median volume particle diameter in the range from 24 to 42 nm and a photogreying index in the range from 0.05 to 3.

2. A metal oxide according to claim 1 wherein the metal oxide particles have a photogreying index in the range from 0.2 to 2.

3. A metal oxide according to claim 1 wherein the metal oxide particles have a yellowing index of less than 6.

4. A metal oxide according to claim 1 wherein the metal oxide particles have a yellowing index in the range from 0.5 to 5.

5. A metal oxide according to claim 1 wherein the metal oxide particles have an extinction coefficient at 524 nm in the range from 0.4 to 1.5 l/g/cm.

6. A metal oxide according to claim 1 wherein the metal oxide particles have an extinction coefficient at 360 nm in the range from 2 to 14 l/g/cm.

7. A metal oxide according to claim 1 wherein the metal oxide particles have an extinction coefficient at 308 nm in the range from 38 to 52 l/g/cm.

8. A metal oxide according to claim 1 wherein the metal oxide particles have at least one of (i) an extinction coefficient at 524 nm in the range from 0.7 to 1.3 l/g/cm, (ii) an extinction coefficient at 450 nm in the range from 1.2 to 1.8 l/g/cm, (iii) an extinction coefficient at 360 nm in the range from 4 to 8 l/g/cm, (iv) an extinction coefficient at 308 nm in the range from 42 to 48 l/g/cm, (v) a maximum extinction coefficient in the range from 61 to 69 l/g/cm, and/or (vi) a λ(max) in the range from 271 to 279 nm.

9. A particulate metal oxide having a median volume particle diameter in the range from 24 to 42 nm and a yellowing index of less than 6.

10. A metal oxide according to claim 9 wherein the metal oxide particles have a yellowing index in the range from 0.5 to 5.

11. A metal oxide according to claim 9 wherein the metal oxide particles have a photogreying index of less than 5.

12. A dispersion comprising metal oxide particles according to claim 1.

13. A dispersion comprising metal oxide particles having an extinction coefficient at 524 nm in the range from 0.4 to 1.5 l/g/cm, a photogreying index in the range from 0.05 to 3, and a yellowing index of less than 6.

14. A sunscreen product comprising metal oxide particles and/or dispersion according to claim 1.

15. The use of metal oxide particles having a median volume particle diameter in the range from 24 to 42 nm and a photogreying index in the range from 0.05 to 3 to produce a sunscreen having reduced photoactivity.

16. The use of a dispersion comprising particles of metal oxide having a median volume particle diameter in the range from 24 to 42 nm and a yellowing index of less than 6, in the manufacture of a transparent sunscreen comprising an organic UV absorber.
